(19) United States
(12) Patent Application Publication
(10) Pub. No.: US 2015/0023898 A1
(43) Pub. Date: Jan. 22, 2015

(54) Composite Particles, Method for Production Thereof, and Use Thereof

(71) Applicant: Eckart GmbH, Hartenstein (DE)

(72) Inventors: Bernd Dressel, Velden (DE); Roland Albert, Offenhausen (DE); Oleg Budakov, Moskau (RU)

(21) Appl. No.: 14/384,522

(22) PCT Filed: Mar. 13, 2013
(86) PCT No.: PCT/EP2013/055166

§ 371 Date: Sep. 11, 2014

(30) Foreign Application Priority Data
Mar. 14, 2012 (DE) 10 2012 102 165.1

(51) Int. Cl. C09C 1/00; A61K 8/81; A61Q 3/02; C09D 5/36

(52) U.S. Cl. CPC C09C 1/0081; C09D 5/36; A61K 8/8152; A61Q 3/02; A61K 8/8123; C09C 2200/507; A61K 2800/10
USPC 424/61; 524/605; 524/567; 524/548; 524/563

(57) ABSTRACT

The present invention relates to composite particles comprising a carrier material as well as at least one pearlescent pigment, wherein the carrier material and the at least one pearlescent pigment are bonded to one another without an adhesive agent. In addition, the invention relates to a method for producing these composite particles as well as the use thereof.

The present invention relates to composite particles, comprising a carrier material and at least one pearlescent pigment, a method for production thereof, and use thereof.

Pearlescent pigments are extremely dust-producing in the dry powdered form. In order to simplify the handling of pearlescent pigments, for example during dosing, pearlescent pigments are supplied in a form moistened with solvent or in a paste-like form. A disadvantage is that in the case of these dosage forms solvents can be carried over into the respective application, for example paints, varnishes, cosmetics, etc.

As alternatives, pearlescent pigment granules are also supplied in which the pearlescent pigments are present with resins, binders, etc. in a form that is bonded, therefore that does not produce dust. A disadvantage in this variant is that agglomerates of pearlescent pigment granules sometimes form which do not completely disintegrate in application. Incompatibilities with the respective application medium can also result because of the resins, binders, etc. used.

DE 10 2008 064 201 A1 relates to non-dust-producing pigment granules which are based on a carrier and are coated with effect pigments by means of an adhesion promoter. Aqueous emulsions, emulsions and dispersions based on acrylated polypropylenes and low-chlorinated polypropylenes or wax emulsions are used as adhesion promoters. Through the emulsion, the carrier material is partially or completely coated with or enveloped by the effect pigment. According to the teaching of DE 10 2008 064 201 A1, the mechanical properties of plastic-based carriers coated with platelet-shaped effect pigments by means of aqueous adhesion promoters are often inadequate, without pre-treatment such as e.g. corona discharge or flame impingement. This manifests itself in particular in the form of dusty wear and loss of adhesion of the coating from the carrier substrate in the form of a delamination. A disadvantage in this variant is also that the adhesion promoters used can lead to incompatibilities in application.

The object of the present invention is to provide pearlescent pigments in a form that produces little or no dust. In particular the pearlescent pigments are to be compatible with the different application media. Furthermore, this dosage form for the pearlescent pigments is to be easily available.

The object of the present invention is achieved by the provision of composite particles which comprise a carrier material and at least one pearlescent pigment, wherein the carrier material and the at least one pearlescent pigment are present bonded to one another without an adhesive agent.

Within the meaning of the invention, by the feature “without an adhesive agent” is meant that the pearlescent pigments are not bonded to the carrier material via a separately applied or arranged adhesive agent or a separately applied or arranged adhesion promoter. The necessity of an adhesion promoter is described for example in DE 10 2008 064 201 A1.

Preferred developments are specified in the dependent claims 2 to 9.

The object of the invention is furthermore achieved by the provision of a method for producing the composite particles according to the invention, wherein the method comprises the following step:

mixing carrier material and at least one pearlescent pigment, wherein the carrier material has an at least partially softened surface.

In addition, a subject of the invention is the use of the composite particles according to the invention to coat substrates, preferably wallpapers or print products, as well as in a coating material or as coating material. The coating material can be paints, printing inks, varnishes and cosmetics. The cosmetics can be nail varnishes, creams, lotions, powders, etc. The cosmetic is preferably a nail varnish.

In particular, the composite particles according to the invention are suitable for the optical enhancement of substrates, in particular wallpapers, print products, etc. The substrates, in particular wallpapers, print products, etc., can be already printed on or provided with patterns.

The inventors have surprisingly found that pearlescent pigments can be provided in a form that is dry and, at the same time, produces little or no dust, if the carrier material and the pearlescent pigments are present bonded to one another without an adhesive agent.

A single pearlescent pigment or several pearlescent pigments can be present bonded to a carrier material. The carrier material is preferably formed particulate. Several pearlescent pigments are preferably bonded to one carrier particle. The at least one pearlescent pigment is, or the pearlescent pigments are, preferably joined to the carrier material in a largely fiat contact. A plurality of pearlescent pigment particles, for example up to 100, up to 250 or up to 500 pearlescent pigments, can be applied and bonded to the carrier material, preferably carrier particle, depending on the size of the carrier particle.

The proportion of pearlescent pigment relative to the total weight of the composite particles preferably lies in a range of from 0.1 to 20 wt.-%, particularly preferably in a range of from 0.2 from 15 wt.-%, further particularly preferably in a range from 0.3 to 12 wt.-% and quite particularly preferably in a range of from 0.5 to 8 wt.-%, in each case relative to the total weight of the composite particles.

Here, the pearlescent pigment lies on the surface of the carrier material and is not enveloped or encapsulated by the latter. In this respect, the composite particle according to the invention differs fundamentally from a pigment preparation for producing masterbatches.

Through the bonding of the pearlescent pigments to the carrier material, preferably carrier particle, the pearlescent pigments can be handled in a low-dust, preferably dust-free manner. Due to the carrier particles, the composite particles have such a high weight that no substantial dust formation, preferably none at all, results during the usual handling of pearlescent pigments, for example during the dosing. In addition, there are preferably no agglomerations in the case of the composite particles according to the invention. Consequently, the composite particles according to the invention are preferably agglomerate-free.

The composite particles according to the invention can extremely advantageously be trickled and/or scattered in a low-dust, preferably dust-free, manner. Thus the composite particles according to the invention can extremely advantageously be scattered for example onto a substrate provided with an adhesive agent and/or binder, for example a wallpaper or a print product, or applied by trickling, without resulting in people or the environment being impacted by a marked formation of dust. Consequently, the composite particles according to the invention supply significant advantages in terms of application technology. Low-dust or dust-free, but still pourable, composite particles are also advantageous when incorporated into a printing ink.

According to a preferred variant of the invention, the pearlescent pigments are material-bonded to at least one surface of the carrier material, preferably the carrier particles. The material-bond is preferably brought about by an at least partial softening of the surface of the carrier material, preferably the carrier particles, followed by adhesion of the pearlescent pigments to the softened area of surface. During the softening of the carrier material, preferably only over part of the surface, the surface becomes sticky, with the result that the pearlescent pigments remain adhered thereto. Once the adhesion of the pearlescent pigments has been carried out, the thus-obtained composite particles are cooled, with the result that the surface of the carrier material, preferably of the carrier particles, solidifies and hardens. Consequently, the pearlescent pigments are bonded to the carrier material, preferably the carrier particles, without a separate adhesive agent. The bonding of the pearlescent pigments is consequently brought about by the carrier material.

The softening of the surface of the carrier material, preferably the carrier particles, is preferably brought about by the application of heat or increasing the temperature. The solidification and hardening of the softened surface of the carrier material, preferably the carrier particles, takes place through cooling, or reducing the temperature. During heating of the carrier material, preferably the carrier particles, not only the surface, but the whole carrier material can soften depending on the chosen temperature and the composition of the carrier material. Of course, this also applies correspondingly to the subsequent cooling process.

Of course, the surface of the carrier material, preferably of the carrier particles, can also be partially dissolved and/or softened by treatment with a solvent, after which a bonding of the pearlescent pigments is made possible, preferably accompanied by drying or volatilization of the solvent.

According to the invention, it is preferred to bring about a softening of the surface of the carrier material, preferably of the carrier particles, by the application of heat.

The bonding of the pearlescent pigments consequently takes place preferably exclusively through the softened and subsequently solidified or hardened carrier material or the material of the carrier particles.

Of course, composite particles according to the present invention can also be graded composite particles which can be obtained for example by sieving or by means of cyclone.

According to a preferred variant of the invention, the carrier material, which is preferably present in the form of carrier particles, is platelet-shaped. By platelet-shaped is meant that the carrier material, preferably the carrier particles, is flat and has an almost uniform, preferably uniform, thickness.

According to a further variant of the invention, the carrier materials, preferably carrier particles, are lens-shaped, spherically or irregularly shaped.

In a preferred embodiment, the carrier materials are present platelet-shaped and polygonal. Further preferably the carrier materials are present tetragonal, pentagonal, hexagonal, heptagonal, octagonal, nonagonal and/or decagonal. Tetragons, hexagons and/or octagons have proved to be very suitable polygons.

According to a further preferred embodiment, the platelet-shaped carrier material, preferably the carrier particles, has a uniform shaping, for example a tetragonal or hexagonal or octagonal shape.

Tetragonal platelet-shaped carrier materials, preferably carrier particles, are rectangles, for example squares, rhombuses, parallelograms or trapeziums.

According to a further preferred development of the invention, the platelet-shaped carrier material, preferably carrier particle, has a hexagonal shape.

According to a further preferred embodiment, the platelet-shaped carrier material, preferably carrier particle, is present as a circular disk or as platelets with a circular circumference.

In a further embodiment, the carrier materials are present in an approximately spherical shape or lens-shaped.

In a further embodiment, the carrier materials are present in an approximately spherical shape.

In a further embodiment, the carrier materials are present approximately lens-shaped.

According to a further variant of the invention, the carrier material, preferably carrier particle, is a plastic. Furthermore, it is preferred that the plastic is a thermoplastic or a thermoplastic elastomer.

The plastic can be a single-variety plastic or a mixture of different plastics, for example different polymers.

Consequently, according to a further variant of the invention, the plastic from which the carrier material, preferably the carrier particles, is produced contains only a portion of thermoplastic or thermoplastic elastomer.

It is advantageous if the plastic used as carrier material, preferably carrier particle, contains thermoplastic or thermoplastic elastomer or preferably consists thereof, as its surface can be softened or partially melted by the application of heat, with the result that the pearlescent pigments can bond to the softened or partially melted surface.

For example polyethylene (PE), polypropylene (PP), polybutylene (PB), polyisobutylene (PI), polystyrene (PS), poly(meth)acrylates, such as e.g. polymethylmethacrylate (PMMA), polyesters, such as e.g. polyethylene terephthalate (PET) and polybutylene terephthalate (PBT), polycarbonate (PC), polyvinyl acetate copolymer (PVAC), polyvinyl chloride (PVC), ethylene acrylic acid copolymer (EAA), polyvinyl acetals, such as e.g. polyvinyl butyrals, polyvinyl butyral (PVB), polyvinyl alcohol (PVAL), polyolefins (PO), polyamide (PA), cellulose acetate (CA), cellulose acetobutyrate (CAB), cellulose nitrate (CN), cellulose triacetate (CTA), ethylene vinyl acetate (EVA), ethylene vinyl acetate copolymer, biodegradable polyesters, such as e.g. polylactic acid (polylactite, PLA), polyethers, such as e.g. polyethylene glycol, polyacrylonitrile or mixtures thereof can be used as suitable plastics or polymers.

Polyesters, such as e.g. polyethylene terephthalate (PET) and polybutylene terephthalate (PBT) as well as polyvinyl acetals, such as e.g. polyvinyl butyrals, polyvinyl butyral (PVB), or mixtures thereof are preferably used as carrier materials, preferably carrier particles.

Polyvinyl butyral (PVB) and polyethylene terephthalate (PET) are particularly preferably used as carrier materials, preferably carrier particles. Polyethylene terephthalate (PET) is quite particularly preferably used as carrier material, preferably carrier particle.

According to a preferred variant of the invention, the plastics soften in a temperature range of from 40 to 230° C., further preferably from 45° C. to 190° C., still further preferably from 50 to 170° C. The softening temperature can also be called the glass transition temperature T_{g}. The glass transition temperature is determined using differential scanning calorimetry (DSC).

Carrier materials with a hexagonal shape and consisting of polyethylene terephthalate (PET) are available for example from RJA Plastics GmbH, 07989 Teichwolframsdorf, Germany, under the trade names Crystal Clear 0.008″ 45.008E, Crystal Clear 0.016″ 45.016E, Crystal Clear 0.025″ 45.025E.

Spherical or lens-shaped carrier particles made of plastic can be produced by first melting the polymer or polymers and, through granulation, such as e.g. underwater granulation, bringing it or them to the desired particle size and shape by means of a perforated disk.

In a further embodiment, the carrier materials, preferably carrier particles, can be present as irregularly shaped polymers. For this, the polymers, as polymer granules, can first be brought to the desired particle size by grinding, e.g. in a pin mill, ball mill or stirred ball mill.

The carrier materials, preferably the carrier particles, are preferably transparent and not dyed. The quantity of light allowed through or transmitted by the carrier materials consists of a directed component and a diffuse component. If the light is scattered in all directions uniformly, this brings about a reduction in the contrast and a milky/cloudy appearance. The so-called haze (clouding) is defined as the quantity of light, in percent, that deviates from the incident light beam on average by more than 2.5° (Byk-Gardner, Qualitätskontrolle für Lacke and Kunststoffe, 2011/2012, p. 61/62). Preferably, in the case of the carrier materials to be used according to the invention, less than 15%, further preferably less than 10%, still further preferably less than 5% of the incident light deviates more than 2.5° from the incident light beam.

To achieve special color effects, carrier materials that are dyed, printed on one side or coated on one side can in addition also be used.

To produce colored platelet-shaped carrier materials, preferably carrier particles, a film can be printed on with a dyed binder, customary in the trade, for solvent-based gravure and flexographic printing inks, preferably before the corresponding film is cut. Polyesters, polyamides, PVC copolymers, aliphatic and/or aromatic ketone resins, melamine-urea resins, melamine-formaldehyde resins, maleinates, colophony derivatives, polyvinyl butyrals, casein or casein derivatives, ethyl cellulose, nitrocellulose and/or aromatic and/or aliphatic polyurethanes or mixtures thereof to which a dyestuff has been added can be used here as dyed binders. Polyvinyl butyral or polyurethane to which up to 20 wt.-% dyestuff, relative to the total weight of the binder, has been added are preferably used as dyed binder.

In a preferred embodiment, colored platelet-shaped carrier materials, preferably platelet-shaped hexagonal carrier particles, are produced by printing for example on a polyethylene terephthalate film with a dyed 25 wt.-% polyvinyl butyral binder using gravure printing, preferably before the polyethylene terephthalate film is cut.

Of course, it is also possible, when producing platelet-shaped carrier materials, preferably carrier particles, for example hexagonal carrier particles, from a film, to use an already colored or dyed film.

In a preferred embodiment, the platelet-shaped carrier material, preferably carrier particle, consists only of a polymer layer, which can optionally be coated with a surface additive.

The two surfaces of the platelet-shaped carrier material, preferably carrier particle, can consist of different materials. The carrier material, preferably carrier particle, in this variant is preferably produced from composite films which can be produced in the conventional manner, for example by coextrusion, lamination or extrusion coating.

Alternatively, one side of the carrier material, preferably in the form of a film, can also be provided with a coating agent or surface additive.

According to the invention, it can be thereby brought about that the pearlescent pigments only bond to one surface of the carrier material, preferably carrier particles. For example, the coating agent or surface additive can prevent the pearlescent pigments from adhering to the coated surface. The coating agent or the surface additive can be applied for example to a film surface before the carrier particles are produced from the film, for example by cutting, stamping, pressing, etc.

Likewise, composite films which are used to produce the carrier material, preferably in the form of carrier particles, can have a film surface to which pearlescent pigments cannot be bonded.

The selective bonding of the pearlescent pigments can be brought about in that the softening temperature of one surface of the carrier material, preferably of the carrier particles, differs from the softening temperature of the other surface, at an applied temperature therefore only one surface softens over the surface and therefore only the softened surface allows a bonding of pearlescent pigments. If the bonding takes place by partial dissolution of the plastic using solvent, a selective bonding to a plastic surface can be set via the different dissolution behavior of the plastic used in the respective layer.

A great advantage in the selective bonding of pearlescent pigments to only one surface of the carrier material, preferably carrier particles, is that the consumption of pearlescent pigments is halved.

Of course, according to a further embodiment of the invention, it is also possible to cover both sides of the carrier material with pearlescent pigment. A covering on both sides can be desired if a very intense pearlescent effect is desired.

Preferably, the carrier material is or the carrier particles are substantially transparent, preferably transparent.

When the composite particles according to the invention are applied to a substrate, for example wallpaper, print product or finger nail, the orientation of the composite particles is almost of no significance in the case of a transparent carrier material. In this case the optical effect of the pearlescent pigments is only slightly influenced by the carrier material.

Platelet-shaped carrier materials with a polygonal shape or a circular shape, preferably a hexagonal or rectangular shape, can be produced by cutting, stamping, pressing, etc. of films or foils made of plastic. A uniform particle size can hereby be guaranteed. Conditional on production, a small proportion of differently shaped or multiply cut particles can also be contained in the carrier material. This proportion, however, is preferably not more than 15 wt.-%, further preferably not more than 10 wt.-%, further preferably not more than 8 wt.-%, still further preferably not more than 5 wt.-%, still further preferably not more than 3 wt.-%, in each case relative to the total weight of carrier material, in order not to impair the optical appearance of the composite particles in an application.

According to a preferred development of the invention, platelet-shaped transparent carrier materials are used which have a polygonal shape, for example a hexagonal or rectangular shape, or a circular shape, wherein the carrier materials can be provided with a coating agent or a surface additive on one side, with the result that the pearlescent pigments do not bond to the coated surface.

In a further embodiment, differently shaped carrier materials, preferably carrier particles, with the same or similar softening temperature can also be used. The pearlescent pigments can thus be applied simultaneously for example to carrier materials in a platelet-shaped hexagonal shape and to carrier materials in an approximately spherical shape. The optical effects which with composite particles which are based on a platelet-shaped hexagonal carrier material as a rule differ from the optical effects which can be achieved with composite particles based on an approximately spherical carrier material. In the case of composite particles based on an approximately spherical carrier material, an observer even outside of the direct top view is faced with more pearlescent pigments than would be the case with composite particles based on a platelet-shaped hexagonal carrier material. For this, composite particles based on an approximately spherical carrier material appear less high-gloss in top view than composite particles which have a platelet-shaped hexagonal carrier material. However, the gloss of composite particles which are based on an approximately spherical carrier material is much less dependent on the viewing angle than the gloss of composite particles which are based on a platelet-shaped hexagonal carrier material is. Of course, in such a comparison, the particle sizes of the pearlescent pigments and the carrier material must be identical or at least comparable.

The particle size of the carrier materials, preferably platelet-shaped carrier particles, preferably lies in a range of from 50 to 2500 μm, further preferably in a range of from 70 to 1600 μm, still further preferably in a range of from 80 to 1000 μm, particularly preferably in a range of from 100 to 800 μm and quite particularly preferably in a range of from 180 to 450 μm.

Approximately spherical, lens-shaped or irregularly shaped carrier materials preferably have a particle-size distribution. The D₅₀ values of these carrier materials preferably lie in a range of from up to 100 to 3000 μm, further preferably in a range of from 120 to 2500 μm, particularly preferably in a range of from 150 to 2000 μm and quite particularly preferably in a range of from 200 to 1300 μm.

If platelet-shaped carrier materials with a polygonal shape, preferably a tetragonal or hexagonal shape, are used, their average thickness preferably lies in a range of from 5 to 150 μm, further preferably in a range of from 6 to 140 μm, particularly preferably in a range of from 7 to 130 μm and quite particularly preferably in a range of from 11 to 120 μm.

If the platelet-shaped carrier material with a polygonal shape has an average thickness of at least 20 μm, the cut edges of the carrier material, in addition to the surface, can also be covered with pearlescent pigments.

If lens-shaped carrier materials are used, their average thickness preferably lies in a range of from 5 to 500 μm, further preferably in a range of from 6 to 450 μm, particularly preferably in a range of from 7 to 370 μm and quite particularly preferably in a range of from 11 to 350 μm. To determine the average thickness of the lens-shaped carrier materials, the respectively largest value for the thickness of each individual particle is determined and, as stated below, averaged over at least 100 particles.

The average thickness of the carrier materials can be determined using scanning electron microscope photographs of polished sections. Here, preferably at least 100 individual particles are measured in order to obtain a meaningful statistic.

To produce the composite particles according to the invention, pearlescent pigments based on non-metallic platelet-shaped substrates are used.

The non-metallic platelet-shaped substrates are preferably substantially transparent, preferably transparent, i.e. they are at least partially, preferably substantially completely, permeable for visible light.

The non-metallic platelet-shaped substrates can be selected from the group consisting of natural mica platelets, synthetic mica platelets, glass platelets, SiO₂ platelets, Al₂O₃ platelets, BiOCl platelets, TiO₂ platelets, Fe₂O₃ platelets, sericite platelets, kaolin platelets, graphite platelets, talc platelets, polymer platelets, platelet-shaped substrates which comprise an inorganic-organic mixed layer. According to the invention, it is also possible to use as pearlescent pigments those having substrates which are mixtures of the platelet-shaped substrates given above.

Preferably, the non-metallic platelet-shaped substrates are selected from the group consisting of natural mica platelets, synthetic mica platelets, glass platelets, SiO₂ platelets, Al₂O₃ platelets and mixtures thereof. Particularly preferably, the non-metallic platelet-shaped substrates are selected from the group consisting of natural mica platelets, synthetic mica platelets, glass platelets and mixtures thereof. Glass platelets and synthetic mica platelets and mixtures thereof are quite particularly preferred as substrates. In particular, glass platelets are preferred as substrate.

Natural mica platelets, unlike synthetic platelet-shaped transparent substrates, have the disadvantage that impurities can alter the hue due to embedded foreign ions, and that the surface is not perfectly smooth due to the production conditions, but can have irregularities, such as e,g. steps.

Synthetic substrates such as for example glass platelets or synthetic mica platelets, on the other hand, can have smooth surfaces as well as a uniform thickness within an individual substrate particle, as well as preferably over the entirety of all substrate particles. Thus, the surface supplies only a few scattering centers for incident or reflected light and thus, after these platelet-shaped substrates have been coated, makes possible higher-gloss and more strongly colored pearlescent pigments than with platelet-shaped natural mica as substrate. Preferably used as glass platelets are any that have been produced according to the methods described in EP 0 289 240 A1, WO 2004/056716 A1 and WO 2005/063637 A1. The glass platelets which can be used as substrate can for example have a composition corresponding to the teaching of EP 1 980 594 B1.

The non-metallic platelet-shaped substrate, as described above, can be provided with at least one layer or coat, wherein the at least one layer preferably comprises metal oxides, metal oxide hydrates, metal hydroxides, metal suboxides, metals, metal fluorides, metal oxyhalides, metal chalcogenides, metal nitrides, metal oxynitrides, metal sulfides, metal carbides or mixtures thereof. According to a preferred variant, the at least one layer or coat consists of the above-named materials.

Metal oxides, metal oxide hydrates, metal hydroxides and/or mixtures thereof are preferably used as the layer or coat. Metal oxides and metal oxide hydrates and/or mixtures thereof are particularly preferably used. The above-named materials can be present either as layers separated from one another or also next to each other in the same layer.

Unless otherwise indicated, the terms layer and coat are used interchangeably.

The coat can either envelop the substrate completely, be only partially present on the substrate, or only cover its upper and/or lower surface.

The at least one layer can be an optically active layer and/or act as a protective layer.

If a high refractive index layer is applied to a non-metallic platelet-shaped substrate, the refractive index lies at n≧1.8, preferably at n≧1.9 and particularly preferably at n≧2.0. In the case of a low refractive index layer or coat, the refractive index lies at n<1.8, preferably at n<1.7 and particularly preferably at n<1.6.

For example, metal oxides such as titanium oxide, preferably titanium dioxide (TiO₂), iron oxide, preferably iron(III) oxide (Fe₂O₃) and/or iron(II/III) oxide (Fe₃O₄), zinc oxide, preferably ZnO, tin oxide, preferably tin dioxide (SnO₂), zirconium oxide, preferably zirconium dioxide (ZrO₂), antimony oxide preferably antimony(III) oxide (Sb₂O₃), magnesium oxide, preferably MgO, cerium oxide, preferably cerium(IV) oxide (CeO₂), cobalt oxide, preferably cobalt(II) oxide (CoO) and/or cobalt(II/III) oxide (Co₃O₄), chromium oxide, preferably chromium(III) oxide (Cr₂O₃), copper oxide, preferably copper(I) oxide (Cu₂O) and/or copper(II) oxide (CuO), vanadium oxide, preferably vanadium(IV) oxide (VO₂) and/or vanadium(III) oxide (V₂O₃), metal sulfides such as zinc sulfide, preferably zinc(II) sulfide (ZnS), metal oxide hydrates such as goethite (FeOOH), titanates such as calcium titanate (CaTiO₃) or iron titanates, such as e.g. ilmenite (FeTiO₃), pseudobrookite (Fe₂TiO₅) and/or pseudorutile (Fe₂Ti₃O₉), doped metal oxides, such as for example titanium dioxide and zirconium dioxide, which are dyed with selectively absorbent dyestuffs and/or mixtures thereof are suitable as the high refractive index layer. The last-named dyeing of non-absorbent high refractive index metal oxides can take place e.g. by incorporating dyestuffs into the metal oxide layer, by doping them with selectively absorbent metal cations or colored metal oxides such as iron(III) oxide or by covering the metal oxide layer with a film containing a dyestuff.

The high refractive index layer preferably comprises metal oxides, metal hydroxides and/or metal oxide hydrates. Metal oxides are particularly preferably used. Titanium dioxide and/or iron oxide as well as mixtures thereof are quite particularly preferably used.

If coating is carried out with titanium dioxide, the titanium dioxide can be present in the rutile or anatase crystal modification. The rutile form can be obtained for example by applying a layer of tin dioxide to the platelet-shaped substrate that is to be coated, for example before the titanium dioxide layer is applied. Titanium dioxide in the rutile modification crystallizes on this layer of tin dioxide. The tin dioxide can be present as a separate layer, wherein the layer thickness can be a few nanometers, for example less than 10 nm, further preferably less than 5 nm, still further preferably less than 3 nm. The tin dioxide, however, can also be present at least partially mixed with the titanium dioxide.

Examples of low refractive index layers are, among others, metal oxides such as silicon oxide, preferably silicon dioxide (SiO₂), aluminum oxide, preferably Al₂O₃, boron oxide, preferably boron(III) oxide (B₂O₃), metal fluorides such as magnesium fluoride, preferably MgF₂, aluminum fluoride, preferably AlF₃, cerium fluoride, preferably cerium(III) fluoride (CeF₃), calcium fluoride, preferably CaF₂, metal oxide hydrates such as aluminum oxide hydrate AlOOH, silicon oxide hydrate, preferably SiO₂.H₂O and/or mixtures thereof.

The low refractive index layer preferably comprises silicon dioxide.

If the non-metallic platelet-shaped substrate is coated with only a single metal oxide layer, the latter preferably has a high refractive index. Depending on the geometric metal oxide layer thickness, such pearlescent pigments can bring about different color effects, as shown in Table 1.

TABLE 1
Typical colors and geometric layer
thicknesses of pearlescent pigments

| | Coverage/geometric | |
|---|---|---|
| | layer thickness | Color |
| Silver white pearlescent | TiO₂: 40-60 nm | silver |
| pigments | | |
| Interference pigments | TiO₂: 20-40 nm | pale blue |
| | TiO₂: 60-80 nm | yellow |
| | TiO₂: 80-100 nm | red |
| | TiO₂: 100-140 nm | blue |
| | TiO₂: 120-160 nm | green |
| | TiO₂: 280-320 nm | green (3rd order) |
| Colored gloss pigments | Fe₂O₃: 35-45 nm | bronze |
| | Fe₂O₃: 45-55 nm | copper |
| | Fe₂O₃: 55-65 nm | red |
| | Fe₂O₃: 65-75 nm | red violet |
| | Fe₂O₃: 75-85 nm | red green |
| | Fe₃O₄ | black |
| Combination pigments | TiO₂/Fe₂O₃ | shades of gold |
| | TiO₂/Cr₂O₃ | green |
| | TiO₂/Prussian blue | dark blue |

If the platelet-shaped transparent substrate consists of synthetic mica, such pearlescent pigments are commercially available e.g. under the trade name Symic (from Eckert, 91235 Hartenstein, Germany). Al₂O₃ flakes coated with TiO₂ and/or iron oxide and correspondingly coated SiO₂ flakes are supplied for example under the trade names Xirallic and Colorstream (from Merck KGaA, Darmstadt, Germany). Glass platelets coated with TiO₂ and/or iron oxide are supplied e.g. under the name Luxan (from Eckart), under the name Firemist (from BASF SE, Ludwigshafen, Germany) or under the name Miraval (from Merck).

Pearlescent pigments based on synthetic substrates and characterized by the values D₁₀, D₅₀ and D₉₀ from the cumulative frequency distribution of the volume-averaged size distribution function with a span ΔD=(D₉₀−D₁₀)/D₁₀ of from 0.7 to 1.4 are characterized, according to WO 2009/103322 A1, by their high color purity. These pearlescent pigments are preferably used according to a preferred variant of the invention.

The non-metallic platelet-shaped substrates can also be coated with a multi-layered layer structure with or consisting of metal oxide, metal hydroxide, metal suboxide and/or metal oxide hydrate, wherein the sequence of the layers can be variable. A layer sequence in which at least one high refractive index layer and at least one low refractive index layer are arranged alternating on a substrate is preferred here. In the case of the alternating arrangement, it is also possible to arrange one or more high refractive index layers directly one on top of the other and then one or more low refractive index layers directly one on top of the other. However, it is essential that high and low refractive index layers occur in the layer structure. Preferably, at least a high, a low and again a high refractive index layer are arranged rising from the platelet-shaped substrate, which results in pearlescent pigments with particularly intense interference colors. The interference color here can be silver or not silver depending on the layer structure and thicknesses.

In an embodiment, the composite particles according to the invention can comprise mixtures of different pearlescent pigments. For example, pearlescent pigments based on e.g. platelet-shaped synthetic mica of different particle sizes or with different coats can be present on the carrier material. In addition, the carrier materials can also be covered with pearlescent pigments based on different substrates, such as e.g. glass platelets and platelet-shaped synthetic mica, with optionally different coats. A carrier material can thus comprise for example both silver and blue pearlescent pigments of the same or different particle size as well as optionally pearlescent pigments based on different platelet-shaped substrates.

The D₅₀ values of the pearlescent pigments to be used according to the invention preferably lie in a range of from 3 to 350 μm, particularly preferably in a range of from 5 to 300 μm and quite particularly preferably in a range of from 6 to 250 μm.

The size-distribution curves of the carrier materials and of the pearlescent pigments are determined using a device from Malvern Instruments GmbH, 71083 Herrenberg, Germany, (Mastersizer 2000) as specified by the manufacturer. For this, the carrier materials or the pearlescent pigments are measured, as an aqueous suspension, several times, preferably three times. Average values are then formed from the individual measurement results. The scattered light signals are evaluated according to the Mie theory. The D₁₀, D₅₀ and D₉₀ values are determined by means of laser diffraction. This means that 10%, 50% and 90%, respectively, of the carrier materials, preferably carrier particles, or effect pigments have a volume-related diameter which is equal to or smaller than the respectively given value.

In a further embodiment, instead of and/or in addition to pearlescent pigments, composite particles according to the invention can also be applied to a carrier material, preferably carrier particle. According to a further embodiment, composite particles, based on a platelet-shaped hexagonal carrier material, and an approximately spherical carrier material can be joined to one another. This happens e.g. by heating the approximately spherical carrier material accompanied by subsequently admixing the composite particle.

If black-white opacity charts are printed on with the composite particles according to the invention, for example using a screen printing process, then an observer initially perceives a high gloss and glitter effect. Outside of the specular angle, because of the transparency of the composite particles according to the invention these black-white opacity charts do not appear dark or black, as would be the case for example with black-white opacity charts printed on with opaque glitters. This angle-dependent gloss effect or so-called light/dark flop can be described objectively by the flop index. The flop index is defined as follows according to Alman (S. Schellenberger, M. Entenmann, A. Hennemann, P. Thometzek, Farbe and Lack, April 2007, p. 130):

flop index=2.69·(L_{E1}−L_{E3})^{1.11}/L_{E2}^{0.88}

where L_{E1} is the lightness of the near-specular measuring angle (E1=15° relative to the specular angle), L_{E2} is the lightness of the measuring angle between near-specular and far-specular angle (E2=45° relative to the specular angle) and L_{E3} is the lightness of the far-specular measuring angle (E3=110° relative to the specular angle). The larger the numerical value of the flop index is, the more greatly the angle-dependent change in lightness, thus the light/dark flop, is expressed.

Under direct illumination, black-white opacity charts which have been printed on or, as in bronzing processes, have been scattered with the composites particles according to the invention for example using a screen printing process appear very glittery. Under diffuse illumination, these black-white opacity charts are perceived as grainy. The graininess is determined using the Byk mac device from Byk-Gardner. To assess the graininess, the high-resolution CCD camera takes a picture with diffuse illumination which is generated by two hemispheres coated white. The image is analyzed with the aid of the histogram of brightness levels, wherein the homogeneity of the light and dark surfaces is summarized in a graininess value. (Byk-Gardner, Qualitätskontrolle für Lacke and Kunststoffe, 2011/2012, p. 98).

The composite particles according to the invention can be obtained using a method which comprises the following step:

mixing carrier material, preferably carrier particles, and at least one pearlescent pigment, wherein the carrier material has an at least partially softened surface.

According to a preferred variant of the invention, the mixing of carrier material, preferably carrier particles, and at least one pearlescent pigment is carried out under heating, wherein the surface of the carrier material, preferably the carrier particles, at least partially softens.

Alternatively, the carrier material, preferably the carrier particles, can also be heated in a first step and then admixed with pearlescent pigment.

Preferably, the heating of the carrier material, preferably the carrier particles, is carried out accompanied by movement of the carrier material.

The movement of the carrier material can take place in a mixer or in a fluidized bed.

According to a preferred variant of the invention, the method according to the invention comprises the following steps:

(a) mixing carrier material and at least one pearlescent pigment, to provide a mixture,
(b) heating the mixture obtained in step (a), to obtain the composite particles,
(c) optionally grading the composite particles obtained in step (b).

According to a preferred variant of the method according to the invention, the mixing in step (a) and preferably also the heating in step (b) are carried out in a mixer, in which the carrier material, preferably the carrier particles, and the at least one pearlescent pigment are moved at a circumferential speed of at least 3 m/s, further preferably at least 4 m/s, still further preferably of at least 6 m/s.

The heating in step (b) takes place depending on the thermal softening properties of the carrier material. In the case of carrier materials made of or with plastics, the softening preferably takes place in a temperature range of from 40 to 230° C., further preferably from 45° C. to 190° C., still further preferably from 50 to 170° C.

An important advantage of this production method is that it can be carried out easily. For the adhesion of the pearlescent pigments to the carrier material, there is no need either to pre-treat the latter in an elaborate manner or to add an additional adhesion promoter. An additional adhesion promoter is not desired according to the invention as the optical properties of the composite particle can be altered in an undesired manner. In addition, there is the danger of incompatibilities of the adhesion promoter with application media. Finally, a separate coating step with an adhesion promoter is elaborate and cost-intensive, in particular in the case of a mass-produced article.

The bonding between carrier material, preferably carrier particle, and the at least one pearlescent pigment is achieved exclusively by specific adhesion, wherein the bonding takes place to the at least partially softened carrier material surface.

In a further embodiment of the invention, the method comprises the following steps:

(a) mixing carrier material and at least one pearlescent pigment, to provide a mixture,
(b) introducing solvent into the mixture obtained in step (a),
(c) volatilizing the solvent out of the mixture treated with solvent in step (b), to obtain the composite particles,
(d) optionally grading, preferably by protective grading, the composite particles obtained in step (b) or (c).

According to a development of the invention, step (b) the solvent can be added as solvent vapor.

According to a development of the invention, step (c) can take place under heating, whereby the volatilization of the solvent is promoted.

In this variant of the method according to the invention, the at least partial softening of the surface of the carrier material, preferably of the carrier material particles, takes place due to the influence of the solvent, whereby the pearlescent pigments bond to the surface.

The composite particles according to the invention are present in a form that does not produce dust, and can be used for dyeing plastic, in nail varnishes or in the graphics industry. In particular, the composite particles according to the invention can be used to enhance wallpapers or print products, such as e.g. greeting cards or folding boxes. This enhancement can take place on the one hand by bronzing, on the other hand in screen or gravure printing processes. An already printed wallpaper or an already printed print product can be given a glittery appearance after bronzing with the composite particles according to the invention. The composite particles according to the invention can also be printed onto already printed wallpapers or print products in screen or gravure printing processes. The printing process with the composite particles according to the invention here can also take place directly during the printing of a desired motif and need not necessarily take place on an already printed wallpaper or print product.

Composite particles based on platelet-shaped carrier materials are preferably applied by means of bronzing, gravure or screen printing. Composite particles based on approximately spherical, lens-shaped or irregularly shaped carrier materials are preferably applied by means of bronzing or scattering devices with engraved scoop rollers. The scoop rollers serve in particular to apply composite particles with an average particle size D₅₀ of from 100 to 2000 μm to the respective substrate in a controlled manner.

The invention furthermore relates to the following preferred embodiments:

Composite particle comprising a carrier material, preferably a platelet-shaped hexagonal carrier particle, as well as at least one pearlescent pigment based on glass platelets, wherein the carrier material and the at least one pearlescent pigment are present bonded to one another without an additional adhesive agent.

Composite particle comprising a carrier material, preferably a platelet-shaped polygonal, preferably hexagonal, carrier particle, as well as at least one pearlescent pigment based on glass platelets or synthetic mica platelets, wherein the carrier material and the at least one pearlescent pigment are present bonded to one another without an additional adhesive agent and the proportion of pearlescent pigment lies in a range of from 0.1 to 20 wt.-%, preferably in a range of from 0.2 to 15 wt.-%, further preferably in a range of from 0.3 to 12 wt.-%, relative to the total weight of the composite particle.

Composite particle comprising a carrier material, preferably a platelet-shaped polygonal, preferably hexagonal, carrier particle made of polyethylene terephthalate (PET), as well as at least one pearlescent pigment based on glass platelets, obtained by mixing carrier material and pearlescent pigment under heating.

Composite particle comprising a carrier material, preferably a platelet-shaped polygonal, preferably hexagonal, carrier particle made of at least two film layers as well as at least one pearlescent pigment based on glass platelets, wherein the carrier material and the at least one pearlescent pigment are present bonded to one another without an additional adhesive agent.

Composite particle comprising a carrier material, preferably a platelet-shaped polygonal, preferably hexagonal, carrier particle made of at least three film layers arranged flat one on top of the other, wherein on the two outer film layers of the carrier material in each case at least one pearlescent pigment is present bonded to the carrier material without an additional adhesive agent.

Composite particle comprising a carrier material made of polyvinyl acetate, preferably an approximately spherical carrier particle, as well as at least one pearlescent pigment based on glass platelets or synthetic mica platelets, wherein the carrier material and the at least one pearlescent pigment are present bonded to one another without an additional adhesive agent.

Composite particle comprising a carrier material made of preferably polyvinyl butyral, preferably a lens-shaped carrier particle, preferably with a particle size of ≦1.5 mm, as well as at least one pearlescent pigment based on glass platelets or synthetic mica platelets, wherein the carrier material and the at least one pearlescent pigment are present bonded to one another without an additional adhesive agent.

Composite particle comprising a first carrier material bonded to a second, preferably spherical, carrier material without an adhesive agent, wherein the first carrier material of the composite particle and the second carrier material are different from one another. The first and the second carrier material are preferably different from one another in terms of the geometric shape. Alternatively or cumulatively, the first carrier material and the second carrier material can also differ in terms of the nature of the substances, preferably the composition.

Composite particle based on a first, platelet-shaped polygonal, preferably hexagonal, carrier particle which is covered with pearlescent pigments based on glass platelets or synthetic mica platelets and wherein the composite particle is bonded to a second carrier material, preferably an approximately spherical carrier particle, preferably made of polyvinyl acetate, with a preferred particle size of ≦2 mm, preferably with a particle size of from 0.5 to ≦1.5 mm, without an adhesive agent. According to a further embodiment of the invention, pearlescent pigments can optionally also be present also on the second, preferably approximately spherical, carrier particle. Coating material such as a cosmetic, preferably a nail varnish, which comprises composite particles.

The following examples and figures serve to describe the invention in more detail and are not intended to be limiting in any respect. All data are to be understood as wt.-%.

### FIGURES

FIG. 1 shows a scanning electron microscope (SEM) photograph of the composite particle from Example 2 magnified 50 times.

FIG. 2 shows a scanning electron microscope (SEM) photograph of the composite particle from Example 2 magnified 200 times.

FIG. 3 shows a scanning electron microscope (SEM) photograph of the composite particle from Example 2 magnified 200 times.

FIG. 4 shows a scanning electron microscope (SEM) photograph of the composite particle from Example 2 magnified 500 times.

FIG. 5 shows a light microscope photograph of the composite particle from Example 1 (scattering on black-white opacity chart) magnified 50 times.

FIG. 6 shows a light microscope photograph of the composite particle from Example 3 (scattering on black-white opacity chart) magnified 50 times.

FIG. 7 shows a scanning electron microscope (SEM) photograph of the composite particle from Example 12 magnified 100 times.

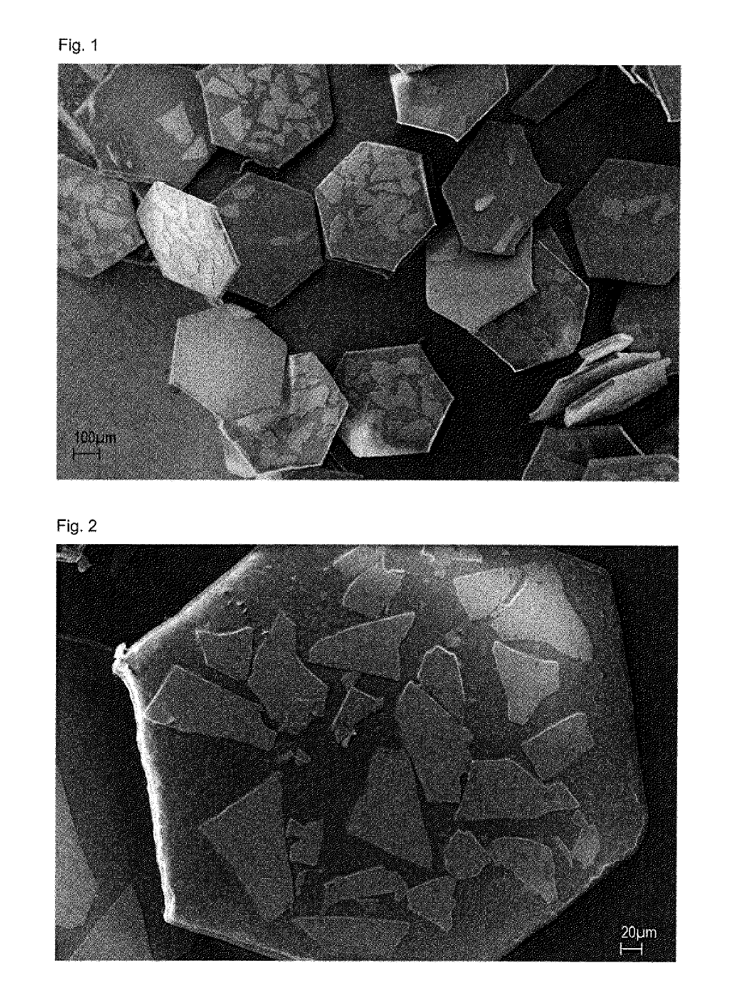

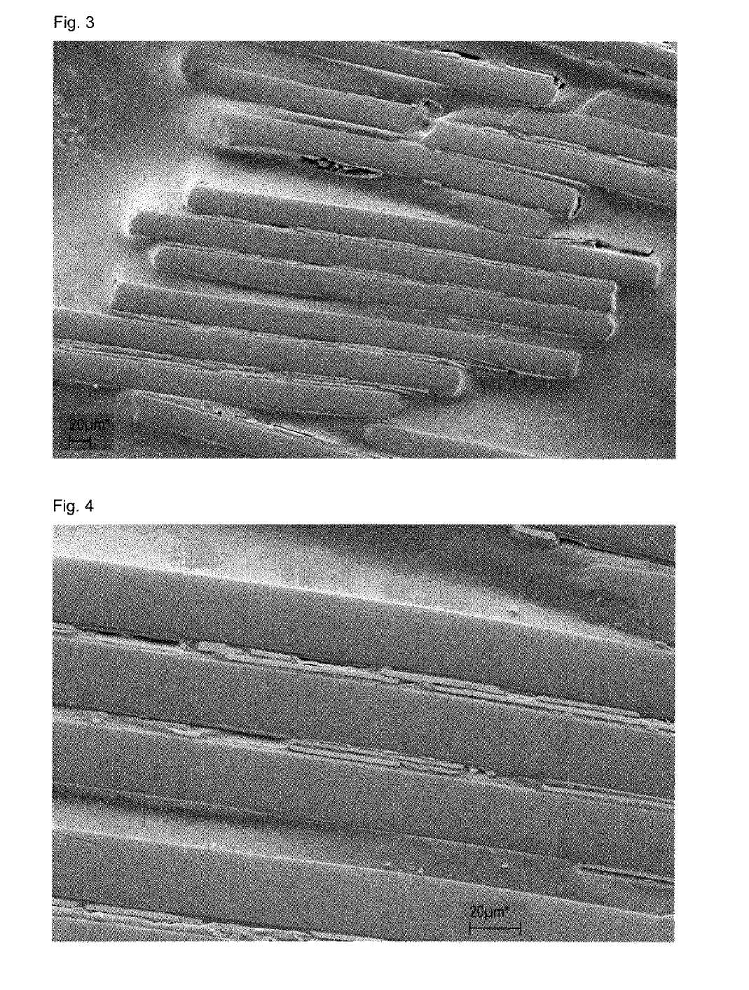

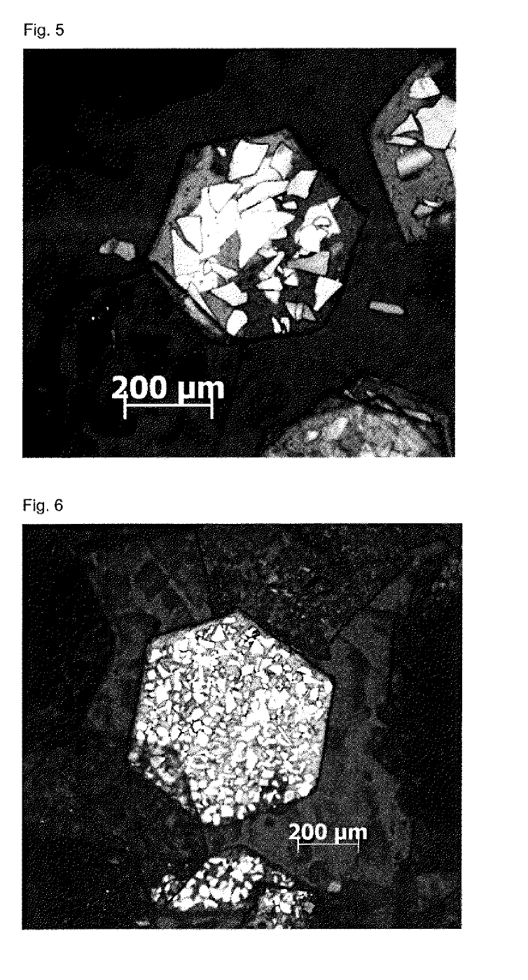

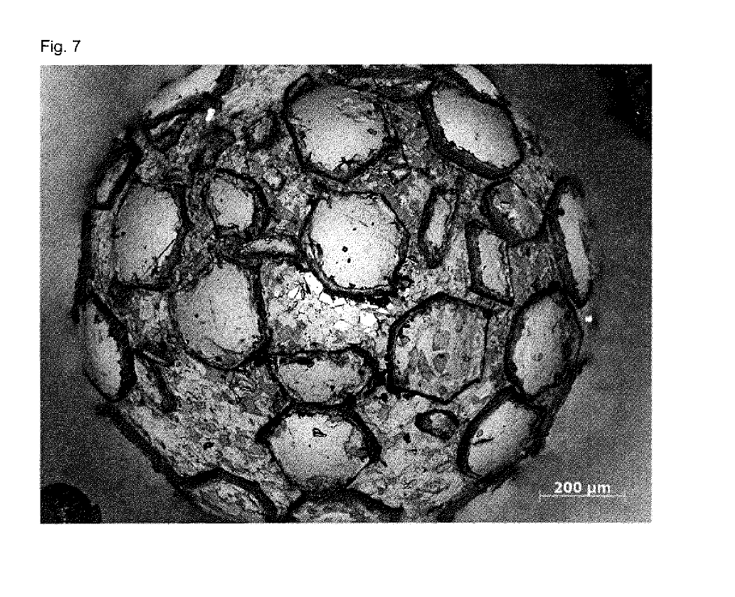

### EXAMPLES

### I Production of the Composite Particles According to the Invention

### Example 1

94 wt.-% PET glitter (Crystal Clear 0.008″ 45.008E, coated on one side to prevent the pearlescent pigments from bonding to the coated side, from RJA plastics GmbH, 07989 Teichwolframsdorf, Germany, particle size as specified by the manufacturer: 200 μm) was placed, together with 5 wt.-% Luxan C001 (D₅₀=30-35 μm) and 1 wt-% Luxan C261 (D₅₀=28-33 μm), both from Eckart, in a Papenmeier laboratory high-speed mixer system, from Gebrüder Lödige Maschinenbau GmbH, 33102 Paderborn, Germany, and mixed for 1 minute at a circumferential speed of 8 m/s. A circumferential speed of 13 m/s was then maintained for 14 minutes, followed by a circumferential speed of 8 m/s for 7 minutes. Before and during the entire mixing process, the mixing tank was heated with a flow temperature of 150° C. After the mixing tank had been emptied, the thus-obtained composite particles were cooled to room temperature and protectively graded via a vibrating sieve with a mesh width of 1000 μm.

### Example 2

94 wt.-% PET glitter (Crystal Clear 0.016″ 45.016E, coated on one side to prevent pearlescent pigments from bonding to the coated side, from RJA plastics GmbH, particle size as specified by the manufacturer: 400 μm) was placed, together with 5 wt-% Luxan E001 (D₅₀=85-90 μm) and 1 wt.-% Luxan E261 (D₅₀=80-85 μm), both from Eckert, in a Papenmeier laboratory high-speed mixer system, from Lödige, and mixed for one minute at a circumferential speed of 8 m/s. A circumferential speed of 13 m/s was then maintained for 19 minutes, followed by a circumferential speed of 8 m/s for 7 minutes. Before and during the entire mixing process, the mixing tank was heated with a flow temperature of 150° C. After the mixing tank had been emptied, the thus-obtained composite particles were cooled to room temperature and protectively graded via a vibrating sieve with a mesh width of 1000 μm.

### Example 3

94 wt.-% PET glitter (Crystal Clear 0.025″ 45.025E, coated on one side to prevent pearlescent pigments from bonding to the coated side, from RJA plastics GmbH, particle size as specified by the manufacturer: 600 μm) was placed, together with 5 wt.-% Luxan C001 (D₅₀=30-35) and 1 wt.-% Luxan C261 (D₅₀=28-33), both from Eckert, in a Papenmeier laboratory high-speed mixer system, from Lödige, and mixed for one minute at a circumferential speed of 8 m/s. A circumferential speed of 13 m/s was then maintained for 22 minutes, followed by a circumferential speed of 8 m/s for one minute. Then the mixing process was interrupted for one minute before a circumferential speed of 8 m/s was set for one minute. This interruption for one minute and subsequent setting of a circumferential speed of 8 m/s for one minute was repeated three times in total. Before and during the entire mixing process, the mixing tank was heated with a flow temperature of 150° C. After the mixing tank had been emptied, the thus-obtained composite particles were cooled to room temperature and protectively graded via a vibrating sieve with a mesh width of 1000 μm.

### Example 4

95 wt.-% PET glitter (Crystal Clear 0.016″ 45.016E, coated on one side to prevent pearlescent pigments from bonding to the coated side, from RJA plastics GmbH) was placed, together with 5 wt.-% Luxan C261 (D₅₀=80-85 μm), from Eckart, in a Papenmeier laboratory high-speed mixer system, from Lödige, and mixed for one minute at a circumferential speed of 8 m/s. A circumferential speed of 13 m/s was then maintained for 19 minutes, followed by a circumferential speed of 8 m/s for 5 minutes. Before and during the entire mixing process, the mixing tank was heated with a flow temperature of 150° C. After the mixing tank had been emptied, the thus-obtained composite particles were cooled to room temperature and protectively graded via a vibrating sieve with a mesh width of 1000 μm.

### Example 5

95 wt.-% PET glitter (Crystal Clear 0.016″ 45.016E, coated on one side to prevent pearlescent pigments from bonding to the coated side, from RJA plastics GmbH) was placed, together with 5 wt.-% Symic C001, from Eckart, in a Papenmeier laboratory high-speed mixer system, from Lödige, and mixed for one minute at a circumferential speed of 8 m/s. A circumferential speed of 13 m/s was then maintained for 25 minutes, followed by a circumferential speed of 8 m/s for 5 minutes. Before and during the entire mixing process, the mixing tank was heated with a flow temperature of 150° C. After the mixing tank had been emptied, the thus-obtained composite particles were cooled to room temperature and protectively graded via a vibrating sieve with a mesh width of 1000 μm.

### Example 6

98 wt.-% PVC granulate (Optigran TG101, from JSC Veika, Vilnius, Lithuania) was placed, together with 2 wt.-% Luxan E001 (D₅₀=85-90 μm), from Eckart, in a Papenmeier laboratory high-speed mixer system, from Lödige, and mixed for one minute at a circumferential speed of 8 m/s. A circumferential speed of 13 m/s was then maintained for 10 minutes, followed by a circumferential speed of 8 m/s for 5 minutes. Before and during the entire mixing process, the mixing tank was heated with a flow temperature of 105° C., After the mixing tank had been emptied, the thus-obtained composite particles were cooled to room temperature and protectively graded via a vibrating sieve with a mesh width of 2000 μm.

### Example 7

97 wt.-% PVC granulate (Optideco 7103 EK1, from JSC VEIKA) was placed, together with 3 wt.-% Luxan F001 (D₅₀=200-210 μm), from Eckart, in a Papenmeier laboratory high-speed mixer system, from Lödige, and mixed for one minute at a circumferential speed of 8 m/s. A circumferential speed of 13 m/s was then maintained for 5 minutes, followed by a circumferential speed of 8 m/s for 5 minutes. Before and during the entire mixing process, the mixing tank was heated with a flow temperature of 120° C. After the mixing tank had been emptied, the thus-obtained composite particles were cooled to room temperature and protectively graded via a vibrating sieve with a mesh width of 1000 μm.

### Example 8

95 wt.-% polyvinyl butyral granulate (PVB granulate B75H 0.4-0.7 mm, from Polikom, Broshniv-Osada, Ukraine) was placed, together with 4 wt.-% Luxan E001 (D₅₀=85-90 μm) and 1 wt.-% Luxan E261 (D₅₀=80-85 μm), both from Eckart, in a Papenmeier laboratory high-speed mixer system, from Lödige, and mixed for one minute at a circumferential speed of 8 m/s. A circumferential speed of 13 m/s was then maintained for 17 minutes, followed by a circumferential speed of 8 m/s for five minutes. Before and during the entire mixing process, the mixing tank was heated with a flow temperature of 120° C. After the mixing tank had been emptied, the thus-obtained composite particles were cooled to room temperature and protectively graded via a vibrating sieve with a mesh width of 2000 μm.

### Example 9

99 wt.-% vinyl acetate homopolymer balls (VINNAPERL 20, from Wacker Chemie AG, Burghausen, Germany, particle-size distribution as specified by the manufacturer 0.1-0.8 mm) were placed, together with 1 wt.-% Phoenix PX 4522, from Eckert, in a Papenmeier laboratory high-speed mixer system, from Lödige, and mixed for one minute at a circumferential speed of 8 m/s. A circumferential speed of 13 m/s was then maintained for 8 minutes, followed by a circumferential speed of 8 m/s for three minutes. Before and during the entire mixing process, the mixing tank was heated with a flow temperature of 90° C. After the mixing tank had been emptied, the thus-obtained composite particles were cooled to room temperature and protectively graded via a vibrating sieve with a mesh width of 2000 μm.

### Example 10

99 wt.-% vinyl acetate homopolymer balls (VINNAPERL 20, from Wacker Chemie AG, Burghausen, Germany, particle-size distribution as specified by the manufacturer 0.1-0.8 mm) were placed, together with 1 wt.-% Luxan 0001, from Eckert, in a Papenmeier laboratory high-speed mixer system, from Lödige, and mixed for one minute at a circumferential speed of 8 m/s. A circumferential speed of 13 m/s was then maintained for 8 minutes, followed by a circumferential speed of 8 m/s for three minutes. Before and during the entire mixing process, the mixing tank was heated with a flow temperature of 90° C. After the mixing tank had been emptied, the thus-obtained composite particles were cooled to room temperature and protectively graded via a vibrating sieve with a mesh width of 2000 μm.

### Example 11

94 wt.-% PET glitter (Crystal Black 0.016″ 104.016E, coated on one side to prevent pearlescent pigments from bonding to the coated side, from RJA plastics GmbH, particle size as specified by the manufacturer: 400 μm) was placed, together with 6 wt.-% Luxan E261 (D₅₀=80-85 μm), from Eckert, in a Papenmeier laboratory high-speed mixer system, from Lödige, and mixed for one minute at a circumferential speed of 8 m/s. A circumferential speed of 13 m/s was then maintained for 19 minutes, followed by a circumferential speed of 8 m/s for 7 minutes. Before and during the entire mixing process, the mixing tank was heated with a flow temperature of 150° C. After the mixing tank had been emptied, the thus-obtained composite particles were cooled to room temperature and protectively graded via a vibrating sieve with a mesh width of 1000 μm.

### Example 12

94 wt.-% PET glitter (Crystal Yellow 0.016″ 50.015E, coated on one side to prevent pearlescent pigments from bonding to the coated side, from RJA plastics GmbH, particle size as specified by the manufacturer: 400 μm) was placed, together with 6 wt.-% Luxan E221 (D₅₀=80-85 μm), from Eckart, in a Papenmeier laboratory high-speed mixer system, from Lödige, and mixed for one minute at a circumferential speed of 8 m/s. A circumferential speed of 13 m/s was then maintained for 19 minutes, followed by a circumferential speed of 8 m/s for 7 minutes. Before and during the entire mixing process, the mixing tank was heated with a flow temperature of 150° C. After the mixing tank had been emptied, the thus-obtained composite particles were cooled to room temperature and protectively graded via a vibrating sieve with a mesh width of 1000 μm.

### Example 13

89 wt.-% vinyl acetate homopolymer balls (VINNAPERL 20, from Wacker Chemie AG, Burghausen, Germany, particle-size distribution as specified by the manufacturer 0.1-0.8 mm) were placed, together with 10 wt.-% PET glitter (Crystal Clear 0.008″, from RJA plastics GmbH, coated on both sides, before the film is cut, with polyvinyl butyral by means of solvent gravure printing with a cylinder configuration of 70 lines/cm and a volume of 14 cm³/m², polyvinyl butyral 25 wt.-% in ethanol), in a Papenmeier laboratory high-speed mixer system, from Lödige, and mixed for one minute at a circumferential speed of 8 m/s. A circumferential speed of 13 m/s was then maintained for 26 minutes. The mixing process was then stopped and 1 wt.-% Luxan C001 (D₅₀=30-35 μm), from Eckart, was added. Then, mixing was carried out for 20 minutes at a circumferential speed of 13 m/s. Before the mixing process and during the mixing process, until the addition of Luxan C001, the mixing tank was heated with a flow temperature of from 110 to 125° C. The mixing tank was then heated with a flow temperature of from 125 to 135° C. until the end of the mixing process. After the mixing tank had been emptied, the thus-obtained composite particles were cooled to room temperature and protectively graded via a vibrating sieve with a mesh width of 2000 μm.

### Example 14

90 wt.-% PET glitter (Crystal Clear 0.08″, from RJA plastics GmbH, coated on both sides, before the film is cut, with polyvinyl butyral by means of solvent gravure printing with a cylinder configuration of 70 lines/cm and a volume of 14 cm³/m², polyvinyl butyral 25 wt.-% in ethanol) was placed, together with 10 wt.-% LUXAN C001, from Eckart, in a Papenmeier laboratory high-speed mixer system, from Lödige, and mixed for one minute at a circumferential speed of 8 m/s. Mixing was then carried out for 12 minutes at a circumferential speed of 13 m/s. Then a circumferential speed of 8 m/s was maintained for 6 minutes. Before and during the mixing process, the mixing tank was heated with a flow temperature of 135° C. In a second method step, 10 wt.-% of the thus-obtained mixture was then placed, with 90 wt-% vinyl acetate homopolymer balls (VINNAPERL 20, from Wacker Chemie AG, Burghausen, Germany, particle-size distribution 0.1-0.8 mm), in a Papenmeier laboratory high-speed mixer system, from Lödige, and mixed for one minute at a circumferential speed of 8 m/s. Then a circumferential speed of 13 m/s was maintained for 29 minutes. A circumferential speed of 8 m/s was then maintained for five minutes. Before and during the mixing process, the mixing tank was heated with a flow temperature of 100° C.

After the mixing tank had been emptied, the thus-obtained composite particles were cooled to room temperature and protectively graded via a vibrating sieve with a mesh width of 2000 μm.

### Example 15

88 wt.-% PET glitter (Crystal Clear 0.016″ 45.016E, from RJA plastics GmbH, particle size as specified by the manufacturer: 400 μm, coated on both sides, before the film is cut, with polyvinyl butyral by means of solvent gravure printing with a cylinder configuration of 70 lines/cm and a volume of 14 cm³/m², polyvinyl butyral 25 wt.-% in ethanol) was placed, together with 10 wt.-% Luxan E001 (D₅₀=85-90 μm) and 2 wt.-% Luxan E261 (D₅₀=80-85 μm), both from Eckart, in a Papenmeier laboratory high-speed mixer system, from Lödige, and mixed for one minute at a circumferential speed of 8 m/s. A circumferential speed of 13 m/s was then maintained for 19 minutes, followed by a circumferential speed of 8 m/s for 7 minutes. Before and during the entire mixing process, the mixing tank was heated with a flow temperature of 150° C. After the mixing tank had been emptied, the thus-obtained composite particles were cooled to room temperature and protectively graded via a vibrating sieve with a mesh width of 1000 μm.

### Comparison Example 1

Polyester hexagon glitter silver 0.008″, from RJA plastics GmbH.

### Comparison Example 2

Polyester hexagon glitter silver 0.016″, from RJA plastics GmbH.

### Comparison Example 3

Polyester hexagon glitter silver 0.025″, from RJA plastics GmbH.

### Comparison Example 4

Crystal Clear 0.008″ 45.008E, coated on one side, from RJA plastics GmbH.

### Comparison Example 5

Crystal Clear 0.016″ 45.016E, coated on one side, from RJA plastics GmbH.

### Comparison Example 6

Crystal Clear 0.025″ 45.025E, coated on one side, from RJA plastics GmbH.

### Comparison Example 7

90 wt.-% PET glitter (Crystal Clear 0.016″ 45.016E, coated on one side to prevent pearlescent pigments from bonding to the coated side, from RJA plastics GmbH) was mixed with 5 wt.-% Luxan C001 (D₅₀=30-35 μm), from Eckart, and homogenized. 10 wt.-% of a 1:1 mixture of Laropal A81, from BASF SE, Ludwigshafen, Germany, and isopropanol were then added and homogenized again. This mixture was scattered onto a sheet in a layer thickness of 3 mm and then dried in a drying oven at 60° C. for 15 min. Due to the easy volatility of the isopropanol the solids content of the mixture after drying lies at 100 wt.-%.

### II Characterization of the Composite Particles According to the Invention

### IIa Particle Size Measurement

The size-distribution curve of the carrier materials and pearlescent pigments was determined using a device from Malvern Instruments GmbH, 71083 Herrenberg (device: Mastersizer 2000) as specified by the manufacturer. For this, approx. 0.1 g of the corresponding carrier material or pearlescent pigment as aqueous suspension, without addition of dispersion auxiliaries, was placed, by means of a Pasteur pipette, accompanied by constant stirring, in the sample preparation chamber of the measuring device and measured three times. The resultant average values were formed from the individual measurement results. The scattered light signals were evaluated according to the Mie theory, which also includes the refraction and absorption behavior of the carrier materials or the pearlescent pigments.

By the average size D₅₀ is meant within the framework of this invention the D₅₀ value of the cumulative frequency distribution of the volume-averaged size distribution function, as obtained by laser diffraction methods. The D₅₀ value indicates that 50% of the carrier materials or pearlescent pigments have a diameter which is equal to or smaller than the given value, for example 20 μm. The D₁₀ and D₉₀ values correspondingly indicate that 10% and 90%, respectively, of the carrier materials or pearlescent pigments have a diameter which is equal to or smaller than the given value.

### IIb Determination of the Average Thickness

The average thickness was determined with the aid of polished sections of the composite particles according to the invention using the Supra 35 scanning electron microscope (from Carl Zeiss AG, 73447 Oberkochen, Germany). In each case at least 100 particles were measured here in order to obtain an informative statistic.

### IIc Scanning Electron Microscope Photographs

The scanning electron microscope photographs were obtained with the aid of polished sections of the composite particles according to the invention using the Supra 35 scanning electron microscope (from Carl Zeiss AG).

### IId Light Microscope Photographs

Light microscope photographs were produced with the aid of black-white opacity charts (Leneta Form 605 C, from Leneta Company, Inc., Mahwah, N.J., USA) with an Axioskop 50 microscope (from Carl Zeiss AG). For this, the particles of the respective examples or comparison examples were scattered onto the black-white opacity charts.

Before the composite particles of the examples according to the invention and the particles according to the comparison examples were scattered, the black-white opacity charts were printed on, by means of screen printing processes (screen: PET 1500 30-120 (threads per cm), from Sefar (9410 Heiden, Switzerland), with the solvent-based screen printing binder Libraspeed LIS (from Marabu GmbH & Co. KG, 71732 Tamm, Germany). For better printability and background wetting, the screen printing binder was provided with 0.25 wt.-% Byk 019 and 0.25 wt.-% Dynwet 800 (both from BYK-Chemie GmbH, 46483 Wesel, Germany), in each case relative to the total weight of the screen printing formulation. The particles of the examples or comparison examples were then scattered at an angle of 45° onto the screen printing binder. Excess effect pigment was knocked off and then dried for 5 min at 80° C. in a drying oven.

### IIe Gloss Measurements

The gloss measurements were carried out with the aid of black-white opacity charts (Leneta Form 605 C, from Leneta) using the micro-TR1-gloss p device (from Byk-Gardner GmbH, 82538 Geretsried, Germany) as specified by the manufacturer, with a measuring geometry of 20°, 60° and 85°, relative to the vertical, in each case on white and black background. A measuring geometry of 60° is suitable for the so-called “medium gloss” in the range of from 10 to 70 gloss points, wherein a higher numerical value of the gloss points corresponds to a higher gloss. A measuring geometry of 20° is suitable for the so-called “high gloss”, i.e. if the gloss value with the measuring geometry of 60° lies at over 70 gloss points, measurements are taken with a measuring geometry of 20°. A measuring geometry of 85° is suitable for the so-called “low gloss”, i.e. if the gloss value with the measuring geometry of 60° lies at less than 10 gloss points, measurements are taken with a measuring geometry of 85°. (Byk-Gardner, catalogue “Qualitätskontrolle für Lacke and Kunststoffe” 2011/2012, p. 16). The gloss values listed below in the table represent average values from five individual measurements in each case.

For this, either the particles of the examples or comparison examples were scattered onto the black-white opacity charts, as described in section IId, or the black-white opacity charts were printed on with them in a screen printing process. For the screen printing, a screen printing ink consisting of 15 wt.-% XY from the examples and comparison examples and 85 wt.-% plastisol PH1046 (from Pharetra Gesellschaft für textile Kunststoffanwendung mbH & Co. KG, 95152 Seibitz, Germany) was produced. This screen printing ink was printed, depending on the particle size, by means of screen printing processes (screen: PET 1500 8-300 (threads per cm/thread thickness in μm) or 21-140 (threads per cm/thread thickness in μm), from Sefar), onto black-white opacity charts and then dried at 170° C. in a drying oven for 1 minute. With a particle size greater than 200 μm the PET 1500 8-300 screen was used, with a particle size of up to 200 μm the PET 1500 21-140 screen was used.

TABLE 2
Gloss values of the particles of the examples or comparison
examples with the aid of the black-white opacity charts from IId

| Example/ | Gloss | | Gloss | | Gloss | |
|---|---|---|---|---|---|---|
| Comparison | 20°, | Gloss 20°, | 60°, | Gloss 60°, | 85°, | Gloss 85°, |
| example | white° | black | white | black | white | black |
| Example 1 | 3.0 | 2.0 | 3.2 | 2.2 | 0.5 | 0.5 |
| Example 2 | 4.6 | 4.8 | 6.4 | 5.0 | 0.6 | 0.5 |
| Example 3 | 8.8 | 7.6 | 16.2 | 14.6 | 2 | 2 |
| Comparison | 6.0 | 4.0 | 5.4 | 3.4 | 0.5 | 0.5 |
| example 4 | | | | | | |
| Comparison | 8.4 | 6.2 | 8.2 | 6.0 | 0.6 | 0.6 |
| example 5 | | | | | | |
| Comparison | 9.8 | 8.2 | 13.2 | 11.6 | 1.9 | 2.0 |
| example 6 | | | | | | |
| Comparison | — | 4.0 | 4.6 | 3.0 | 0.6 | 0.5 |
| example 7 | | | | | | |

TABLE 3
Gloss values of the particles of the examples or comparison examples,
which were printed onto black-white opacity charts in a screen printing
process.

| Example/ | Gloss | | Gloss | | Gloss | |
|---|---|---|---|---|---|---|
| Comparison | 20°, | Gloss 20°, | 60°, | Gloss 60°, | 85°, | Gloss 85°, |
| example | white° | black | white | black | white | black |
| Example 1 | 12.8 | 9.2 | 53.0 | 47.0 | 70.6 | 74.4 |
| Example 2 | 7.8 | 6.2 | 44.0 | 40.4 | 71.8 | 71.8 |
| Example 3 | 7.8 | 6.0 | 41.8 | 39.0 | 51.8 | 57.2 |
| Comparison | 11.8 | 15.0 | 48.8 | 56.6 | 56.4 | 69.4 |
| example 1 | | | | | | |
| Comparison | 7.6 | 7.2 | 38.8 | 38.6 | 51.6 | 65.2 |
| example 2 | | | | | | |
| Comparison | 11.4 | 9.4 | 46.2 | 44.4 | 59.0 | 60.8 |
| example 4 | | | | | | |
| Comparison | 8.2 | 5.0 | 43.2 | 35.2 | 60.6 | 61.0 |
| example 5 | | | | | | |
| Comparison | 5.4 | 3.6 | 24.8 | 25.4 | 30.6 | 39.4 |
| example 6 | | | | | | |
| Comparison | 4.2 | 3.6 | 23.8 | 24.8 | 27.2 | 31.8 |
| example 7 | | | | | | |

### IIf Determination of the Tight/Dark Flop (Flop Index)

The light/dark flop (flop index) was determined with the aid of the black-white opacity charts from IIe, which were printed on with particles of the examples or comparison examples in a screen printing process, using the BYK-mac device (from Byk-Gardner). The flop index is defined as follows according to Alman (S. Schellenberger, M. Entenmann, A. Hennemann, P. Thometzek, Farbe and Lack, April 2007, p. 130):

flop index=2.69·(L_{E1}−L_{E3})^{1.11}/L_{E2}^{0.86}

wherein L_{E1} stands for the lightness of the near-specular measuring angle (E1=15° relative to the specular angle), L_{E2} for the lightness of the measuring angle between near-specular and far-specular angle (E2=45° relative to the specular angle), and L_{E3} for the lightness of the far-specular measuring angle (E3=110° relative to the specular angle).

The values listed in the table below were measured on the white background of the black-white opacity chart.

### IIg Graininess

The graininess G was determined with the aid of the black-white opacity charts from section IIe, which were printed on with the particles of the examples or comparison examples in a screen printing process, using the BYK-mac device (from Byk-Gardner).

To assess the graininess, the high-resolution CCD camera takes a picture with diffuse illumination which is generated by two hemispheres coated white. The image is analyzed with the aid of the histogram of brightness levels, wherein the homogeneity of the light and dark surfaces is summarized in a graininess value. The higher this value is, the grainier the black-white opacity chart printed on with particles of the examples or comparison examples appears in the case of diffuse illumination. The higher the graininess of a black-white opacity chart printed on with the particles of the examples or comparison examples is under diffuse light, the more this is visually perceived to be glittery under direct illumination (Byk-Gardner, is Qualitätskontrolle für Lacke and Kunststoffe, 2011/2012, p. 98).

TABLE 4
Lightness L*, flop index and graininess G

| Example/ | | | | | | | |
|---|---|---|---|---|---|---|---|
| Comparison | | | | | L*, | Flop | |
| example | L*, 15° | L*, 25° | L*, 45° | L*, 75° | 110° | index | G |
| Example 1 | 100.7 | 95.1 | 92.4 | 91.8 | 89.1 | 0.8 | 3.3 |
| Example 2 | 100.7 | 94.3 | 89.8 | 90.2 | 87.9 | 1.0 | 4.8 |
| Example 3 | 101.9 | 94.2 | 90.3 | 89.2 | 87.2 | 1.1 | 3.4 |
| Comparison | 110.1 | 96.4 | 86.7 | 75.2 | 73.6 | 3.1 | 22.5 |
| example 1 | | | | | | | |
| Comparison | 104.2 | 90.8 | 72.9 | 65.7 | 59.4 | 4.6 | 23.6 |
| example 2 | | | | | | | |
| Comparison | 103.5 | 77.8 | 61.1 | 58.3 | 57.0 | 5.6 | 22.6 |
| example 3 | | | | | | | |

The larger the numerical value of the flop index is, the more greatly the light/dark flop is expressed. When Examples 1 to 3 are compared, it is to be recognized that the value of the flop index increases as the particle size of the composite particles increases. The same is also true for comparison examples 1 to 3. A comparison of Examples 1 to 3 with comparison examples 1 to 3 shows that in the latter the value of the flop index is much higher. In a visual assessment of the black-white opacity charts printed on with the comparison examples 1 to 3 these also lose their metallic luster outside of the specular angle and appear dark to an observer. This optical effect is much less pronounced in the case of Examples 1 to 3, which is explained by the transparency of the composite particles according to the invention. Because of this transparency, the white background of the black-white opacity chart shines through outside of the specular angle.

The higher the numerical value G of the graininess is, the grainier the black-white opacity chart printed on with the respective example or comparison example appears to an observer under diffuse light. In a comparison of Examples 1 to 3 with comparison examples 1 to 3, much higher values for the graininess G are to be observed in the latter. This is also visible in a visual assessment of the respective black-white opacity charts. Comparison examples 1 to 3, in contrast to Examples 1 to 3, show a much higher, irregularly acting glitter effect, while a more homogeneous appearance is perceived in the case of Examples 1 to 3.

### III Application-Specific Examples

### Application-Specific Example 1

### Application of Example 1 to a Wallpaper in a Screen Printing Process

For the screen printing, a screen printing ink consisting of 15 wt.-% of the composite particles from Example 1 and 85 wt.-% plastisol PH1046 (from Pharetra) was produced. This screen printing ink was printed, by means of screen printing processes (screen: PET 1500 18-180 (threads per cm/thread thickness in μm), from Sefar), onto an already printed wallpaper and then dried at 200° C. in a drying tunnel.

### Application-Specific Example 2

### Application of Example 2 to a Wallpaper in a Scattering Process

To enhance a wallpaper already printed on by means of gravure printing processes, it was printed on with an adhesive layer of Optifoam, from Veika, in a gravure printing process. The composite particles from Example 2 were then applied to this wallpaper by means of a scattering unit. After the wallpaper had been dried in the drying tunnel at 150° C., excess composite particles were removed by suction.

### Application-Specific Example 3

### Application of Example 1 to a Wallpaper in an Aqueous Gravure Printing Process

For the gravure printing, a gravure printing ink consisting of 15 wL-% of the composite particles from Example 1 and 85 wt.-% Rotostar Aqua 400 255 medium, from Eckart, was produced and set to a printing viscosity of 30 s with water in a DIN 4 flow cup, from Byk-Gardner. This gravure printing ink was printed, by means of gravure printing processes, onto an already printed wallpaper and then dried at 150° C. in a drying tunnel.

### Application-Specific Example 4

### Nail Varnish

| INCI name | Product name | wt.-% | Manufacturer |
|---|---|---|---|
| | Example 1 | 3 | |
| Butylacetate, Ethylacetate, | International | 98.00 | International |
| Nitrocellulose, Isopropyl | Lacquers Base | | Lacquers |
| Alcohol | 12616 | | |

### Application-Specific Example 5

### Nail Varnish

| INCI name | Product name | wt.-% | |
|---|---|---|---|
| | Example 3 | 4 | |
| Butylacetate, Ethylacetate, | International | 96.00 | International |
| Nitrocellulose, Isopropyl | Lacquers Base | | Lacquers |
| Alcohol | 12616 | | |

### Application-Specific Example 6

### Nail Varnish

| INCI name | Product name | wt.-% | |
|---|---|---|---|
| | Example 2 | 2 | |
| | Syncrystal Silver | 2 | Eckart |
| Butylacetate, Ethylacetate, | International | 96.00 | International |
| Nitrocellulose, Isopropyl | Lacquers Base | | Lacquers |
| Alcohol | 12616 | | |

1. A composite particle comprising a carrier material and at least one pearlescent pigment,
wherein the carrier material and the at least one pearlescent pigment are present bonded to one another without an adhesive agent, and wherein the pearlescent pigment lies on the surface of the carrier material and is not enveloped or encapsulated by the carrier material.

2. The composite particle according to claim 1,
wherein the carrier material has a glass transition temperature.

3. The composite particle according to claim 1,
wherein the carrier material is platelet-shaped.

4. The composite particle according to claim 1,
wherein the carrier material is approximately spherical, lens-shaped or irregularly shaped.

5. The composite particle according to claim 1,
wherein the carrier material has a polygonal shape.

6. The composite particle according to claim 1,
wherein the at least one pearlescent pigment is joined to the carrier material in a largely flat contact.

7. The composite particle according to claim 1,
wherein the at least one pearlescent pigment and the carrier material are material-bonded to one another.

8. The composite particle according to claim 1,
wherein the composite particle comprises a first carrier material, wherein the first carrier material is bonded to a second carrier material without an adhesive agent, wherein the second carrier material and the first carrier material of the composite particle are different from one another.

9. The composite particle according to claim 1,
wherein the carrier material is a plastic.

10. The composite particle according to claim 9,
wherein the plastic is a thermoplastic or thermoplastic elastomer.

11. A method for producing a composite particle according to claim 1,
the method comprising:
mixing carrier material and at least one pearlescent pigment, wherein the carrier material has an at least partially softened surface.

12. A process for producing a coated substrate comprising coating a substrate with the composite particle according to claim 1.

13. (canceled)

14. A coating material
comprising composite particles according to claim 1.

15. The coating material according to claim 14,
wherein the coating material is a paint, printing ink, varnish or cosmetic.

16. (canceled)

17. The composite particle according to claim 1, wherein the carrier material comprises polyethylene, polypropylene, polybutylene, polyisobutylene, polystyrene, poly(meth)acrylate, polyester, polycarbonate, polyvinyl acetate copolymer, polyvinyl chloride, ethylene acrylic acid copolymer, polyvinyl acetal, polyvinyl alcohol, polyolefin, polyamide, cellulose acetate, cellulose acetobutyrate, cellulose nitrate, cellulose triacetate, ethylene vinyl acetate, ethylene vinyl acetate copolymer, biodegradable polyester, polyether, polyacrylonitrile and mixtures thereof.

18. The composite particle according to claim 1, wherein the carrier material is platelet-shaped.

19. The composite particle according to claim 1, wherein two surfaces of the platelet-shaped carrier material consists of two different materials.

20. The composite particle according to claim 1, wherein one side of the carrier material is provided with a coating agent or surface additive.

21. The composite particle according to claim 1, wherein a selected surface of the carrier material has the at least one pearlescent pigment bonded thereto.

22. The composite particle according to claim 1, wherein both sides of the carrier material have the at least one pearlescent pigment bonded thereto.

23. The composite particle according to claim 1, wherein less than 15% of the incident light reflected from a surface of the carrier material deviates on average by more than 2.5° from an incident light beam.

24. The composite particle according to claim 1, wherein the at least one pearlescent pigment comprises a non-metallic substrate selected from the group consisting of natural mica platelets, synthetic mica platelets, glass platelets, SiO2 platelets, Al2O3 platelets, BiOCl platelets, TiO2 platelets, Fe2O3 platelets, sericite platelets, kaolin platelets, graphite platelets, talc platelets, polymer platelets, platelet-shaped substrates which comprise an inorganic-organic mixed layer, and mixtures thereof.

25. The process according to claim 12, wherein the substrate is wallpaper or a print product.

26-28. (canceled)
